(19) United States
(12) Patent Application Publication
(10) Pub. No.: US 2016/0007855 A1
(43) Pub. Date: Jan. 14, 2016

(54) IMAGE DETECTION SYSTEM FOR DIAGNOSING PHYSIOLOGIC STATUS OF ORGAN HAVING FLUORESCENT MATTER

(71) Applicant: National Taiwan University, Taipei (TW)

(72) Inventor: Tzu-Ming Liu, Taipei (TW)

(73) Assignee: National Taiwan University, Taipei (TW)

(21) Appl. No.: 14/624,002

(22) Filed: Feb. 17, 2015

(30) Foreign Application Priority Data
Jul. 9, 2014 (TW) 103123583

(51) Int. Cl. A61B 5/00; A61K 49/00

(52) U.S. Cl. CPC A61B 5/0071; A61B 5/0064; A61B 5/0075; A61K 49/0034; A61B 5/0068; A61B 2576/00; A61B 2562/0233

(57) ABSTRACT

An image detection system for diagnosing a metabolic function and physiological status of a tissue or an organ having a fluorescent matter is provided. The image detection system includes a source emitting exciting light, a scan unit converting the exciting light into a scan of the exciting light, a light-guiding unit including an object lens, a splitter unit, and a detection unit. The splitter unit directs the scan of the exciting light to enter a tissue having a fluorescent matter such that the fluorescent matter in the tissue is excited by the scan of the exciting light and emits fluorescence light, whereby a layer of the tissue is scanned by the scan of the exciting light. The splitter unit directs the fluorescence light emitted from the tissue to the detection unit for processing so as to generate a fluorescent image of the layer of the tissue.

### CROSS-REFERENCE TO RELATED APPLICATION

This application claims foreign priority under 35 U.S.C. §119(a) to patent application Ser. No. 10/312,3583, filed on Jul. 9, 2014, in the Intellectual Property Office of Ministry of Economic Affairs, Republic of China (Taiwan, R.O.C.), the entire content of which patent application is incorporated herein by reference.

### BACKGROUND OF THE INVENTION

1. Field of the Invention

This invention relates to fluorescence molecules image detection systems, and, more particularly, to an image detection system for diagnosing a metabolic function and physiologic status of a tissue or an organ having a fluorescent matter.

2. Description of Related Art

Hepatocellular carcinoma (HCC) is one of the most common cancers and is also one of the leading causes of death worldwide. The incidence of HCC originates from the cirrhosis related to the chronic viral hepatitis, and surgical liver resection is one of the means for the treatment of HCC. To avoid liver failure after surgery and to enable the liver after surgery to play role in body metabolism, in preoperative assessment, an indocyanine green (ICG) retention test will be applied on patients to evaluate the hepatic functions. This ICG retention test can help doctors to evaluate how much hepatic functions remains in patients and determine the portion of liver resection.

Generally, for healthy people, the residual ICG on 15 minutes post administration (ICG-R15) will be below 15%. In contrast, the retention level could be above 40% for patients at the late stage of HCC. So far, the procedure of ICG-R15 invasively takes a draw of blood at the 10 or 15 minutes post intravenous administration. The initial concentration is calculated and estimated through the body weight of patient and the amount of ICG injected. However, this method is not a continuous measurement. Initial concentration maybe changed with individual differences. Besides, some patients still have allergic response to the ICG at the regulated concentration.

In order to achieve continuously monitoring the retention of ICG, a technique as referred pulse dye densitometry (PDD) has been developed by Nihon Kohedn. In this technique, blood absorbance changes are detected through the skin with an optical probe using a variation of pulse oximetry principles. This variation improves on the prior technique by eliminating the necessity for repeated blood withdrawal. However, as described above, this technique remains limited by the difficulty of separating absorbance changes due to the dye concentration changes from absorbance changes due to changes in blood oxygen saturation or blood content in the volume of tissue interrogated by the optical probe. This technique is also expensive in requiring large amounts of dye to create noticeable changes in absorbance and a light source producing two different wavelengths of light for measuring light absorption by the dye and hemoglobin differentially. Furthermore, this technique is a single point measurement method, in which there are melanin absorption and diffraction phenomena since the measurement range covers skin, such that the sensitivity for sensing ICG is decreased.

Therefore, how to overcome the above problems to provide a sensitive and accurate technology for diagnosing metabolic function and physiologic status of a tissue or an organ is a critical issue to be resolved.

### SUMMARY OF THE INVENTION

The present invention provides an image detection system for diagnosing a metabolic function and physiological status of an organ having a fluorescent matter, comprising: a source for emitting exciting light; a scan unit for converting the exciting light into a scan of the exciting light; a light-guiding unit including an object lens, the object lens guiding the scan of the exciting light into a tissue having a fluorescent matter such that the fluorescent matter in the tissue is excited by the scan of the exciting light and emits fluorescence light, whereby a layer of the tissue is scanned by the scan of the exciting light; a detection unit detecting and processing the fluorescence light emitted by the tissue, so as to generate a fluorescent image of the layer of the tissue; a splitter unit for directing the scan of the exciting light to the object lens to enter the tissue, and further directing the fluorescence light emitted by the tissue to the detection unit such that the fluorescent image of the layer of the tissue is generated by the detection unit.

In an embodiment, the detection unit comprises a filter, a photomultiplier and a processor. The filter allows fluorescence light having a particular wavelength to pass through and reach the photomultiplier, and the photomultiplier receives and converts the fluorescence light having the particular wavelength into an electric signal, such that the processor processes the electric signal and generates the fluorescent image of the layer of the tissue.

In another embodiment, the splitter unit reflects the scan of the exciting light to the object lens to enter the tissue, and further transmits the fluorescence light emitted by the tissue to the detection unit. Alternatively, the splitter unit transmits the scan of the exciting light to the object lens to enter the tissue, and further reflects the fluorescence light emitted by the tissue to the detection unit.

The present invention further provides an image detection system for diagnosing a metabolic function and physiological status of a tissue or an organ having a fluorescent matter, comprising: a source for emitting exciting light; a scan unit for converting the exciting light into a scan of the exciting light; a light-guiding unit including an object lens, the object lens guiding the scan of the exciting light into a tissue having a fluorescent matter such that the fluorescent matter in the tissue is excited by the scan of the exciting light and emits fluorescence light, whereby a layer of the tissue is scanned by the scan of the exciting light; a detection unit for detecting and processing the fluorescence light emitted by the tissue, so as to generate a fluorescent image of the layer of the tissue; and a splitter unit for directing the exciting light to the scan unit such that the exciting light is converted into the scan of the exciting light by the scan unit, and further directing the fluorescence light emitted by the tissue to the detection unit after the scan of the exciting light enters the tissue via the object lens, such that the fluorescent image of the layer of the tissue is generated by the detection unit.

In an embodiment, the detection unit comprises a filter, a confocal optics, a photomultiplier and a processor. The filter allows the fluorescence light having a particular wavelength emitted by the tissue to pass through and reach the photomultiplier, and the confocol optics allows the fluorescence light having the particular wavelength emitted by the layer of the tissue to pass through and reach the photomultiplier, so as for the photomultiplier to receive and convert the fluorescence light with the particular wavelength emitted by the layer of the tissue into an electric signal, and then for the processor to process the electric signal and generate the fluorescent image of the layer of the tissue.

In another embodiment, the splitter unit transmits the exciting light to the scan unit, and further reflects the fluorescence light emitted by the tissue to the detection unit. Alternatively, the splitter unit reflects the exciting light to the scan unit, and further transmits the fluorescence light emitted by the tissue penetrating to the detection unit.

In an embodiment, the fluorescent matter is indocyanine green (ICG), bilirubin, flavin, or bilirubin oxidant. When the fluorescent matter is ICG, the image detection system according to the present invention employs the exciting light having a wavelength in a range of 760 nm-800 nm; and when the fluorescent matter is bilirubin or the bilirubin oxidant, the image detection system according to the present invention employs the exciting light having a wavelength in a range of 400 nm-650 nm. Furthermore, the fluorescence light emitted by ICG has a wavelength in a range of 820 nm-850 nm, the fluorescence emitted by the bilirubin has a wavelength in a range of 530 nm-550 nm, and the fluorescence emitted by the bilirubin oxidant has a wavelength in a range of 655 nm-685 nm.

Therefore, the image detection system according to the present invention determines a metabolic function and physiological status of a tissue or an organ by means of a noninvasive continuous image monitoring process, such that a quantization error in the single point measurement can be avoided and a more sensitive and accurate fluorescent matter retention rate can be obtained, which as a diagnostic basis of organ resection in preoperative assessment.

### BRIEF DESCRIPTION OF THE DRAWINGS

The invention can be more fully understood by reading the following detailed description of the preferred embodiments, with reference made to the accompanying drawings.

FIG. 1A is a schematic diagram of an image detection system for diagnosing a metabolic function and physiological status of a tissue or an organ having a fluorescent matter of an embodiment according to the present invention.

FIG. 1B is a schematic diagram of an aspect of the embodiment of FIG. 1A.

FIG. 2 is a schematic diagram of an image detection system for diagnosing a metabolic function and physiological status of a tissue or an organ having a fluorescent matter of another embodiment according to the present invention.

FIGS. 3A and 3B are a fluorescent image and a relative fluorescence intensity decay curve of an ear tissue of a rat having a normal hepatic functions obtained by using the present invention, respectively.

FIG. 3C is a relative fluorescence intensity decay curve of an ear tissue of a rat having a liver tumor obtained by using the present invention.

FIG. 4 is a schematic diagram of the fluorescence intensity emitted by the flavins, bilirubins, and bilirubin oxidants measured by using the present invention.

### DETAILED DESCRIPTION OF THE INVENTION

The following illustrative embodiments are provided to illustrate the disclosure of the present invention, these and other advantages and effects can be apparently understood by those in the art after reading the disclosure of this specification. The present invention can also be performed or applied by other different embodiments. The details of the specification may be on the basis of different points and applications, and numerous modifications and variations can be devised without departing from the spirit of the present invention.

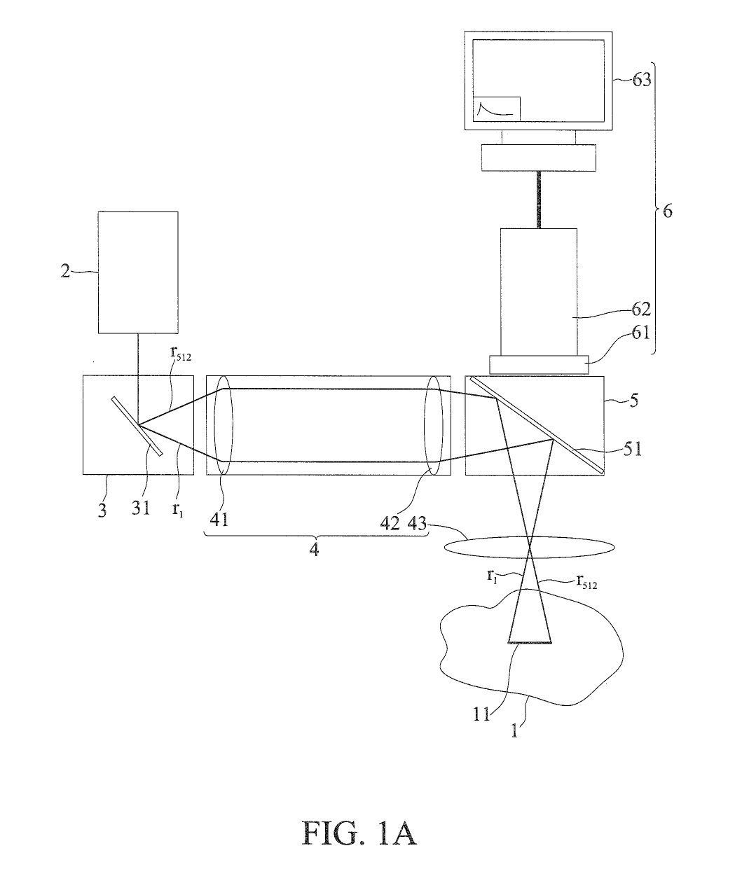

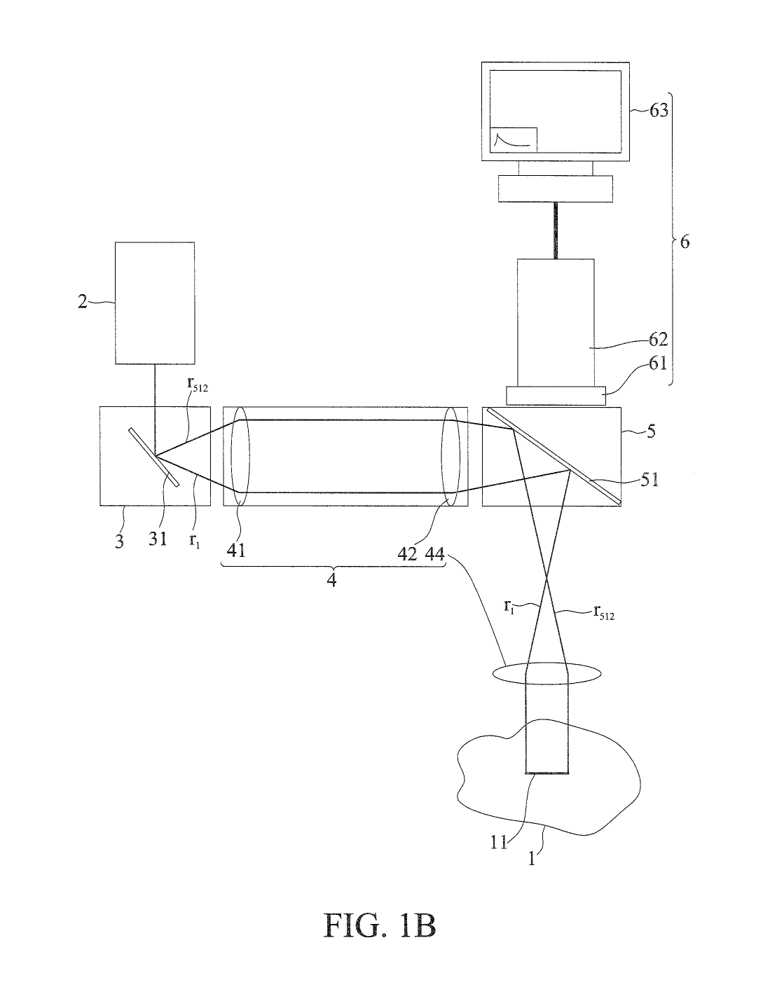

Referring to FIGS. 1A and 1B, an image detection system for diagnosing a physiological status (e.g., a metabolic function) of an organ or a tissue located in the organ having a fluorescent matter according to the present invention detects a fluorescent molecules image for a tissue 1. It should be noted that the image detection system according to the present invention may diagnose physiological status of an organ, such as a metabolic function of a liver, or an oxygen concentration and its change of a tissue so as to know whether the tissue located in an organ is anoxic or not.

An image detection system according to the present invention comprises a source 2, a scan unit 3, a light-guiding unit 4, a splitter unit 5, and a detection unit 6.

The tissue 1, for example, is a tissue in a human arm or in ears of an animal. The fluorescent matter, such as indocyanine green (ICG), flavins, bilirubin, bilirubin oxidant, or biliverdin, is injected into a vein in advance or endogenously generated such that the tissue 1 includes the fluorescent matter. The ICG, when excited, emits fluorescence light having a wavelength ranging about 820 nm-850 nm. The bilirubin & flavins, when excited, emits fluorescence light having a wavelength ranging about 530 nm-550 nm, and the bilirubin oxidant, when excited, emits fluorescence light having a wavelength ranging about 655 nm-685 nm.

The source 2 emits exciting light to the scan unit 3. For ICG, the exciting light is near infrared laser that has a wavelength ranging about 633 nm-800 nm, preferably 760 nm-800 nm, or femtosecond Cr: forsterite laser having a central wavelength of 1230 nm. For the bilirubin or the bilirubin oxidant, the exciting light has a wavelength ranging about 400 nm-650 nm. When the exciting light is directed to the tissue 1, the fluorescent matter in the tissue 1 is excited by the exciting light and emits fluorescence light.

The scan unit 3 comprises a rotating mirror 31 that rotates periodically. The scan unit 3 receives the exciting light from the source 2, and the periodically rotating rotating mirror 31 converts the exciting light into a scan of the exciting light. In other words, the scan unit 3 can output a plurality of beams of exciting ray of light sequentially in different directions, e.g., r1-r512 (only exciting rays of light r1 and r512 are illustrated in FIGS. 1A and 1B), to scan the tissue 1.

The light-guiding unit 4 comprises, but not limited to, two lens 41 and 42 (or called “relay lens”) and an object lens (for focusing) 43. The splitter unit 5 comprises a beam splitter 51. The scan of the exciting light from the scan unit 3 is guided to the beam splitter 51 by the relay lens, then reflected to the object lens 43 by the beam splitter 51, and finally focused onto the tissue 1 by the object lens 43 so as to scan one layer of the tissue 1. It should be noted that though all regions which are excited by the scan of the exciting light in the tissue 1 can emit the fluorescence light, only one layer on the focal plane of the object lens 43 in the tissue 1 can be excited more efficiently to emit the fluorescence light, as compared with other layers outside the focal plane, such that the scan of the exciting light can be considered merely to scan one layer of the tissue 1 through the object lens 43. In addition, the fluorescence light emitted from the fluorescent matter in the tissue 1 excited by the scan of the exciting light is directed to the beam splitter 51 by the object lens 43, and further transmitted to the detection unit 6 by the beam splitter 51.

The detection unit 6 comprises a filter 61, a photomultiplier 62 and a processor 63. The filter 61 allows fluorescence light having a particular wavelength to pass through and reach the photomultiplier 62. The particular wavelength has a range depending on the type of the fluorescent matter. For example, if the fluorescent matter is ICG, the image detection system employs the filter 61 which allows the fluorescence light having a wavelength in a range of 820 nm-850 nm to pass. For example, if the fluorescent matter is flavin or bilirubin, the image detection system employs the filter 61 which allows the fluorescence light having a wavelength in a range of 530 nm-550 nm to pass. For example, if the fluorescent matter is bilirubin oxidants, the image detection system employs the filter 61 which allows the fluorescence light having a wavelength in a range of 655 nm-685 nm to pass. The photomultiplier 62 receives the fluorescence light having the particular wavelength passed by the filter 61 and converts the fluorescence light into an electrical signal. The processor 63 processes the electrical signal and generates a fluorescent image of the one layer of the tissue 1, which is referred to as an optical section.

Furthermore, in the embodiment of FIG. 1, if the fluorescent matter is ICG, the beam splitter 51 reflects the scan of the exciting light having a wavelength in a range of 760 nm-800 nm, and transmits the fluorescence light having a wavelength in a range of 820 nm-850 nm, such that the scan of the exciting light from the scan unit 3 can be reflected to the tissue 1 by the beam splitter 51, and the fluorescence light emitted from the tissue 1 can be transmitted to the detection unit 6 by the beam splitter 51. In another embodiment, if the fluorescent matter is ICG, the beam splitter 51 can transmit the scan of the exciting light having a wavelength in a range of 760 nm-800 nm and reflect the fluorescence light having a wavelength in a range of 820 nm-850 nm, and the tissue 1 and the detection unit 6 also vary their positions. Besides, the range of the wavelength of the fluorescence light which the beam splitter 51 reflects or transmits depends on the type of the fluorescent matter.

In addition, as showed FIG. 1B, the object lens 43 also can be replaced with an f-theta lens 44, which has a focal plane with nearly zero curvature. Using the f-theta lens 44 has a benefit of obtaining a fluorescent image of a layer of the tissue 1 with more perfect optical quality.

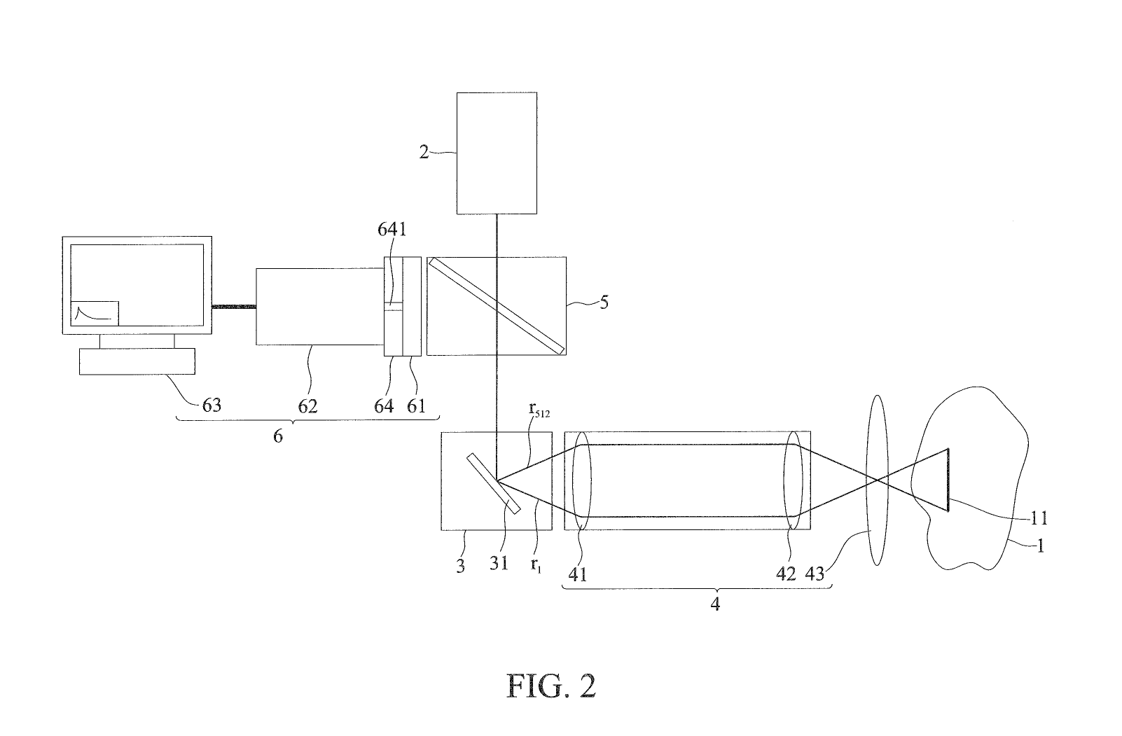

Furthermore, referring to FIG. 2, an embodiment of an image detection system for diagnosing a metabolic function and physiological status of a tissue or an organ having a fluorescent matter according to the present invention is illustrated. The difference between the embodiments in FIG. 2 and FIG. 1 is that the detection unit 6 further comprises a confocal optics 64 and the splitter unit 5 changes its position.

The confocal optics 64 is a plate having a pinhole to filter the fluorescence light which is not excited by the layer on the focal plane in the tissue 1.

The source 2 emits exciting light to the splitter unit 5, and the splitter unit 5 transmits the exciting light to the scan unit 3 to be converted into a scan of the exciting light. The scan of the exciting light is guided by the light-guiding 4 to the tissue 1 to form a focal plane in the tissue 1 so as to scan a layer 11 on the focal plane in the tissue 1.

Next, the fluorescent matter in the tissue 1 is excited by the scan of the exciting light so as to emit fluorescence light. The fluorescence light is guided by the light-guiding unit 4 and reflected by the scan unit 3 to the splitter unit 5 sequentially, and then reflected by the splitter unit 5 to the detection unit 6 to be processed such that a fluorescent image of the layer 11 of the tissue 1 is generated. For the detection unit 6, the filter 61 only allows fluorescence light having a particular wavelength to pass through and reach the photomultiplier 62. For example, if the fluorescent matter is ICG, the image detection system employs the filter 61 which allows fluorescence light having a wavelength in a range of 820 nm-850 nm to pass. For example, if the fluorescent matter is bilirubin or flavin, the image detection system employs the filter 61 which allows fluorescence light having a wavelength in a range of 530 nm-550 nm to pass. For example, if the fluorescent matter is bilirubin oxidant, the image detection system employs the filter 61 which allows fluorescence light having a wavelength in a range of 655 nm-685 nm to pass. The confocal optics 64 merely allows the fluorescence light emitted by the layer 11 on the focal plane in the tissue 1 to enter the photomultiplier 62. The photomultiplier 62 receives the fluorescence light having the particular wavelength emitted from the layer 11 of the tissue 1, and converts the fluorescence light into an electrical signal. The processor 63 processes the electrical signal, and generates a fluorescent image of the layer 11 of the tissue 1, which is referred to as an optical section. It should be noted that other layers outside the focal plane are also excited by the scan of the exciting light to emit fluorescence light when the scan of the exciting light scans the one layer 11 of the tissue 1. The confocal optics 64 in this embodiment can block the fluorescence light which is not emitted from the one layer 11 such that the fluorescent image of the one layer of the tissue can be generated by the processor 63.

Furthermore, in the embodiment in FIG. 2, if the fluorescent matter is ICG, the splitter unit 5 transmits the exciting light having a wavelength in a range of 760 nm-800 nm and reflects the fluorescence light having a wavelength in a range of 820 nm-850 nm. Thus, the exciting light from the source 2 can be transmitted by the splitter unit 5 to the scan unit 3 to be converted into the scan of the exciting light by the scan unit 3 such that the scan of the exciting light is guided by the light-guiding unit 4 to enter the tissue 1. In addition, the fluorescence light emitted from the tissue 1 can be reflected to the detection unit 6 by the splitter unit 5. In another embodiment, if the fluorescent matter is ICG, the splitter unit 5 can reflect the exciting light having a wavelength in a range of 760 nm-800 nm and transmit the fluorescence light having a wavelength in a range of 820 nm-850 nm, and the tissue 1 and the detection unit 6 also vary their positions. Besides, the object lens 43 in this embodiment also can be replaced with the f-theta lens 44, and the range of the wavelength of the fluorescent light that the splitter unit 5 reflects or transmits depends on the type of the fluorescent matter.

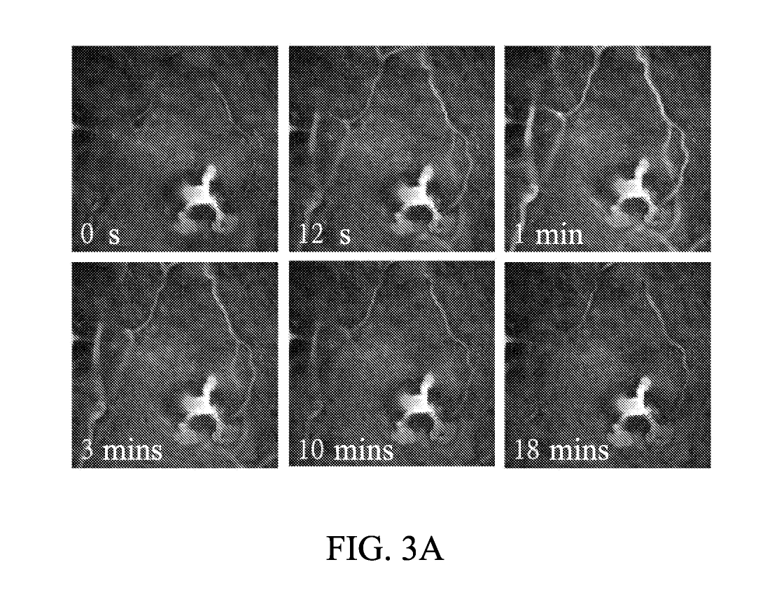

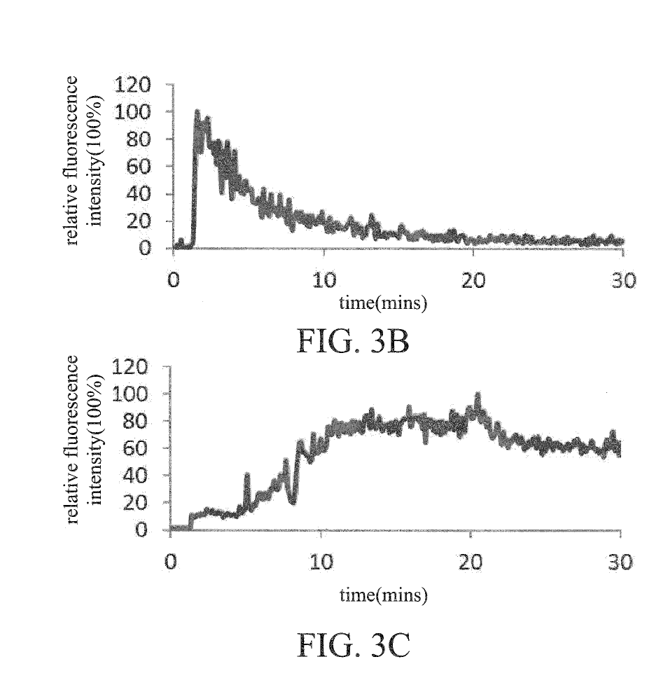

Refer to FIGS. 3A and 3B, which are a fluorescent image and a relative fluorescence intensity decay curve of a tissue in a rat with a normal hepatic functions by injecting ICG into a vein of a rat tail, respectively. FIG. 3C shows a relative fluorescence intensity decay curve of a tissue in a rat with a liver tumor by injecting ICG into a vein of a rat tail. In this embodiment, a region containing a vein of a rat ear is excited using laser at 780 nm, and the fluorescence light emitted from the ICG is gathered. Background fluorescence noises do not exist, since the fluorescence light emitted from ICG has a wavelength in a range of 820 nm-850 nm, which is greater than self-emitting red fluorescence light at about 680 nm. FIG. 3A shows real time fluorescent images of a tissue in a rat with a normal hepatic functions at different times after ICG is injected. Moreover, in comparison of FIG. 3B with FIG. 3C, the ICG retention level is above 40% for a rat with a liver tumor.

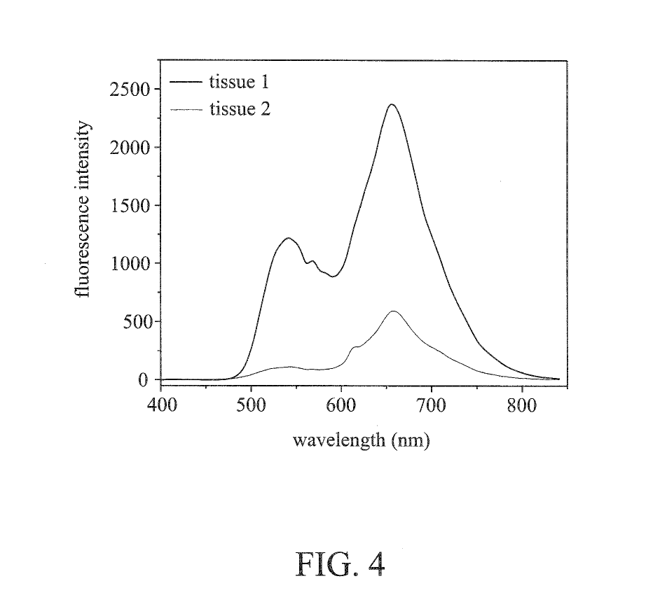

Furthermore, refer to FIG. 4 and Table 1. FIG. 4 depicts the intensity of the fluorescence light emitted from the tissue 1 (wide line) and tissue 2 (narrow line). Table. 1 represents the relationship between the concentration of bilirubins in blood plasma and the intensity of the fluorescence light having a wavelength of 657 nm-657 nm. Besides, the fluorescence light having a wavelength of 540 nm is emitted by bilirubin, and the fluorescence light having a wavelength of 657 nm is emitted by bilirubin oxidant or derivative.

TABLE 1

| | Fluorescence | Fluorescence | |
|---|---|---|---|
| | light intensity | light intensity | Bilirubins |
| Tissue | (540 nm) | (657 nm) | (mg/dl) |
| 1 | 1222 | 2376 | 59.62 |
| 2 | 113 | 593 | 5.33 |

According to table 1 and FIG. 4, when the bilirubin concentration is up to about 10 times, the intensity of fluorescence light having a wavelength of about 540 nm is increased. That is, the fluorescence light intensity at 540 nm is proportional to the concentration of bilirubin in blood plasma. Since the bilirubin is one of pigment in bile, which can be used as an important basis for the determination of jaundice in clinical, and an important indicator for hepatic and gallbladder function, measuring the decay of the fluorescence intensity emitted by the tissue can understand the status of the organ for metabolism such as liver or gall.

In summary, the present invention excites the fluorescent matter in the tissue by using laser having a wavelength in a range of 760 nm-800 nm or 400 nm-650 nm, and then detects the fluorescence light emitted by the fluorescent matter in the tissue to quantify the concentration of the fluorescent matter so as to continuously measure the variation of the concentration of the fluorescent matter, and further computes the fluorescent matter retention rate such that errors due to quantify the concentration of the fluorescent matter by means of absorbance changes are excluded. Moreover, the present invention scans one layer of the tissue by using the scan unit, e.g., a vein in the one layer of the tissue, so as to determine that the fluorescence light comes from the vein of the one layer rather than being self-emitting fluorescence light comes from other layers, thereby enabling a more accurate concentration quantification for the fluorescent matter. Furthermore, the present invention focuses the laser onto the tissue by the light-guiding unit to excite the fluorescent matter in the vein so as to avert from a scattering interference produced by the epidermal tissue, thereby achieving highly efficient excitation for the fluorescent matter.

The foregoing descriptions of the detailed embodiments are only illustrated to disclose the features and functions of the present invention and not restrictive of the scope of the present invention. It should be understood to those in the art that all modifications and variations according to the spirit and principle in the disclosure of the present invention should fall within the scope of the appended claims.

What is claimed is:

1. An image detection system, comprising:
a source that emits exciting light;
a scan unit that converts the exciting light into a scan of the exciting light;
a light-guiding unit including an object lens, the object lens guiding the scan of the exciting light into a tissue having a fluorescent matter such that the fluorescent matter in the tissue is excited by the scan of the exciting light and emits fluorescence light, whereby a layer of the tissue is scanned by the scan of the exciting light;
a detection unit that detects and processes the fluorescence light emitted by the tissue, so as to generate a fluorescent image of the layer of the tissue; and
a splitter unit that directs the scan of the exciting light to the object lens so as to enter the tissue, and further directs the fluorescence light emitted by the tissue to the detection unit such that the fluorescent image of the layer of the tissue is generated by the detection unit.

2. The image detection system of claim 1, wherein the detection unit comprises a filter, a photomultiplier and a processor, the filter is used to allow the fluorescence light having a particular wavelength to enter the photomultiplier, so as for the photomultiplier to receive and convert the fluorescence light into an electric signal, and then for the processor to process the electric signal and generate the fluorescent image of the layer of the tissue.

3. The image detection system of claim 1, wherein the splitter unit reflects the scan of the exciting light to the object lens such that the scan of the exciting light enters the tissue via the object lens, and further transmits the fluorescence light emitted by the tissue to the detection unit.

4. The image detection system of claim 1, wherein the splitter unit transmits the scan of the exciting light to the tissue, and further reflects the fluorescence light emitted by the tissue to the detection unit.

5. The image detection system of claim 1, wherein the exciting light has a wavelength in a range of 400 nm-650 nm or 760 nm-800 nm.

6. The image detection system of claim 1, wherein the fluorescence light emitted by the tissue has a wavelength in a range of 820 nm-850 nm, 530 nm-550 nm or 655 nm-685 nm.

7. The image detection system of claim 1, wherein the fluorescent matter is indocyanine green, bilirubins, flavins, or bilirubin oxidants.

8. The image detection system of claim 1, wherein the object lens is an f-theta lens.

9. An image detection system, comprising:
a source that emits exciting light;
a scan unit that converts the exciting light into a scan of the exciting light;
a light-guiding unit including an object lens, the object lens guiding the scan of the exciting light into a tissue having a fluorescent matter such that the fluorescent matter in the tissue is excited by the scan of the exciting light and emits fluorescence light, whereby a layer of the tissue is scanned by the scan of the exciting light;
a detection unit that detects and processes the fluorescence light emitted by the tissue, so as to generate a fluorescent image of the one layer of the tissue; and
a splitter unit that directs the exciting light to the scan unit such that the exciting light is converted into the scan of the exciting light by the scan unit, and further directs the fluorescence light emitted by the tissue to the detection unit after the scan of the exciting light enters the tissue via the object lens, such that the fluorescent image of the layer of the tissue is generated by the detection unit.

10. The image detection system of claim 9, wherein the detection unit comprises a filter, a confocal optics, a photomultiplier and a processor, the filter is used to allow the fluorescence light having a particular wavelength emitted by the tissue to enter the photomultiplier, and the confocol optics is used to allow the fluorescence light having the particular wavelength emitted by the one layer of the tissue to reach the photomultiplier, so as for the photomultiplier to receive and convert the fluorescence light having the particular wavelength emitted by the layer of the tissue into an electric signal, and then for the processor to process the electric signal and generate the fluorescent image of the layer of the tissue.

11. The image detection system of claim 9, wherein the splitter unit transmits the exciting light to the scan unit, and further reflects the fluorescence light emitted by the tissue to the detection unit.

12. The image detection system of claim 9, wherein the splitter unit reflects the exciting light to the scan unit, and further transmits the fluorescence light emitted by the tissue to the detection unit.
